SEQUENCE LISTING (8 sequences)

SEQ ID NO: 1 - DNA, 60 bp, Artificial Sequence
  Primer
ggccagtgaattgtaatacgactcactatagggaggcggttttttttttttttttttttt

SEQ ID NO: 2 - DNA, 36 bp, Artificial Sequence
  Splint Oligonucleotide
ggcgcgcctttttttttttttttttttttttttttt

SEQ ID NO: 3 - DNA, 19 bp, T7
taatacgactcactatagg

SEQ ID NO: 4 - DNA, 19 bp, SP6
atttaggtgacactataga

SEQ ID NO: 5 - DNA, 20 bp, T3
aattaaccctcactaaaggg

SEQ ID NO: 6 - DNA, 76 bp, Artificial Sequence
  Synthetic Hairpin Oligonucleotide
gaggcgcgcccatttaatgggcgcgcctctaatacgactcactatagggagatttttttttttttttttttttttt

SEQ ID NO: 7 - DNA, 24 bp, Artificial Sequence
  Synthetic Primer Oligonucleotide
tttttttttttttttttttttttt

SEQ ID NO: 8 - DNA, 75 bp, Artificial Sequence
  Synthetic Splint Oligonucleotide- T7 Asc
aaaaaaaaaaaaaaaaaaaaaaaaccctatagtgagtcgtattaggcgcgccggnggnggnggnggnggnggacc

(19) United States
(12) Patent Application Publication
(10) Pub. No.: US 2006/0166245 A1
(43) Pub. Date: Jul. 27, 2006

(54) Rolling circle amplification of micro-RNA samples

(76) Inventors: S. Steven Potter, Cincinnati, OH (US); Hung-Chi Liang, Mason, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 11/331,589

(22) Filed: Jan. 13, 2006

Related U.S. Application Data
(63) Continuation of application No. PCT/US04/22997, filed on Jul. 16, 2004.
(60) Provisional application No. 60/487,972, filed on Jul. 17, 2003.

(51) Int. Cl. C12Q 1/68; C12P 19/34

(52) U.S. Cl. 435/6; 435/91.2

(57) ABSTRACT

The compositions of the present invention find use in amplifying RNA obtained from subjects, particularly very small RNA samples. The methods allow conversion of RNA into circularized cDNA suitable for amplification by rolling circle replication. The amplified cDNA is then transcribed into RNA resulting in amplified RNA.

### CROSS-REFERENCE TO RELATED APPLICATIONS

This application is a continuation of, and claims priority to and benefit of, PCT/US04/022997, filed on Jul. 16, 2004, which application claimed priority to U.S. Provisional Application No. 60/487,972, filed on Jul. 17, 2003, the disclosures of each are herein incorporated by reference in their entirety.

### GOVERNMENT GRANT INFORMATION

This invention was made with Government support under NIH Grant No. 1R01DR61916-01. The United States Government has certain rights in this invention.

### FIELD OF THE INVENTION

The present invention relates to the field of molecular biology, more particularly to the amplification of RNA and expression analysis.

### BACKGROUND OF THE INVENTION

Expression analysis is critical to understanding normal and abnormal development, disease progression, and even to determining the presence or absence of a disease state. One of the most common methods of expression analysis involves the use of microarrays. Microarrays allow the simultaneous analysis of transcript levels for multiple genes. It is even possible to define the gene expression level of the entire complement of genes in tissues. Microarrays are used to study the expression of thousands of genes in various tissues. Expression analysis using microarrays typically requires 5-10 micrograms of total RNA. Large tissue samples or cultured cells readily yield 10 micrograms of RNA; however, total RNA in a single cell is estimated to be less than 20 picograms. Thus, gene expression analysis of small samples is inhibited by the RNA requirements. However, very small samples, such as samples obtained from embryonic organs, needle biopsies of diseased or tumor tissue, or isolated single cells, can be the most interesting. Small samples present a more precise snapshot of gene expression than that presented by analysis of large tissue samples.

There are currently two primary methods used for amplification of small RNA samples. One is the reverse transcription-polymerase chain reaction, or RT-PCR. The RT-PCR procedure makes cDNA. Known sequences are added to the cDNA ends, sometimes by ligation. The presence of known sequence at the cDNA ends allows PCR amplification of the cDNA. Each PCR cycle amplifies the DNA two-fold, and multiple PCR cycles can be performed efficiently. The PCR method tends to amplify short sequences more efficiently than long sequences. Additionally, PCR poorly amplifies some nucleotide sequences, such as GC-rich sequences. With each PCR cycle the inefficient or poor amplification of a nucleotide sequence in a prior cycle affects the relative proportions of the nucleotide sequences in the sample. Some sequences become more relatively abundant than others. The compounding effects of the high number of cycles required for PCR amplification can be quite significant.

The second method of RNA amplification is serial in vitro transcription. Each transcription cycle yields 200 to 500 fold amplification. However, each round of the in vitro transcription method requires synthesis of double-stranded cDNA. Random primers are used to initiate synthesis of the second strand. The use of random primers tends to result in under-representation of the 5′ ends of the products. Multiple in vitro transcription cycles increase the 5′ to 3′ bias of the sample.

Thus, development of a method for amplifying RNA is desirable for use in expression analysis. The method should allow the amplified RNA to reflect the original relative abundance of the RNA transcripts. It is of importance to develop a method of amplifying RNA for analysis of expression in small samples. It is of particular importance to develop a method of amplifying RNA for analysis of complex tumor tissues, embryonic material, and cellular responses.

### SUMMARY OF THE INVENTION

The present invention provides methods and compositions for the amplification of RNA and comparison of RNA expression levels in multiple samples. The invention allows efficient conversion of RNA into circular cDNA templates suitable for rolling circle amplification. Amplified cDNA can then be labeled using any one of a variety of methods. In an embodiment the amplified cDNA is transcribed into amplified RNA.

The compositions of the present invention find use in amplifying RNA obtained from subjects, particularly very small RNA samples. The methods allow conversion of RNA into circularized cDNA suitable for amplification by rolling circle replication. The amplified cDNA is then transcribed into RNA resulting in amplified RNA.

In an embodiment, the invention provides methods for amplifying RNA. The methods involve providing RNA; synthesizing cDNA with predetermined nucleotide sequences at the 3′ ends of the cDNA from the RNA; incubating the cDNA with a splint oligonucleotide; circularizing the cDNA; and preparing amplified cDNA from the circularized cDNA using rolling circle replication. The methods of the invention involve incubating RNA with a primer. Isolated RNA anneals with a primer of the invention. A primer of the invention is an isolated nucleic acid molecule comprising a poly(dT) region. In aspects of the invention, the primer further comprises a promoter region. In an embodiment of the invention, the nucleotide sequence of the primer is set forth in SEQ ID NO:1. In an embodiment of the invention, the nucleotide sequence of the primer is set forth in SEQ ID NO:7. In an embodiment of the invention, the promoter region of the primer comprises the T7 promoter (SEQ ID NO:3), the SP6 promoter (SEQ ID NO:4), or a nucleotide sequence having at least about 90% sequence identity to SEQ ID NO:3 or SEQ ID NO:4. The nucleotide sequence of the promoter region is capable of initiating transcription. Typically, the promoter region is 5′ to the middle region, and the middle region is 5′ to the poly(dT) region. The middle region varies in length as described elsewhere herein. The poly(dT) region is located at the extreme 3′ end of the primer.

In an embodiment, synthesizing cDNA comprises a first strand synthesis step. In the method, the RNA and primer mixture is incubated with a reverse transcriptase. A primer of the invention introduces predetermined nucleotide sequence at the 5′ end of the cDNA. Any available reverse transcriptase, including but not limited to, moloney murine leukemia virus reverse transcriptase, avian myeloblastosis virus reverse transcriptase, recombinant Thermus thermophilus DNA polymerase, and Expand reverse transcriptase may be used in the method of the invention. Reverse transcriptase extends the primer to generate an RNA-cDNA mixture. The cDNA has predetermined nucleotide sequence at the 5′ ends. In an embodiment, the RNA and cDNA mixture is incubated with a ribonuclease H (RNAseH). RNAseH removes RNA duplexed to cDNA. The cDNA may be purified at one or more stages in the methods of the invention. In an aspect of the invention, the reaction mixture is incubated with a 3′ to 5′ exonuclease.

In an embodiment, the linear cDNA produced by first strand synthesis is incubated with terminal transferase. Terminal transferase modifies a 3′ terminus of the cDNA by adding a first homopolymer region. The first homopolymer region is at least 5 homogenous nucleotides. The predominant nucleoside of the first homopolymer region may be deoxyadenosine, deoxyguanosine, deoxycytidine, or thymidine. In an embodiment, the first homopolymer region is adenosine. After the terminal transferase reaction, the linear single-stranded cDNAs have predetermined sequence at both the 5′ and 3′ ends.

In an aspect of the invention, a splint oligonucleotide is incubated with the cDNA. Splint oligonucleotides are isolated nucleic acid molecules comprising a second homopolymer region and a variable region. Typically the second homopolymer region is 3′ to the variable region. The nucleoside of the second homopolymer region is complementary to the nucleoside of the first homopolymer region. The second homopolymer region is at least 5 nucleotides. In an embodiment the variable region's nucleotide sequence contains a palindrome. In an embodiment, the nucleotide sequence of the variable region includes a restriction enzyme site. In an embodiment, the nucleotide sequence of the variable region includes a promoter region. In an embodiment, the variable region includes a primer complementary region and a central region. The primer complementary region is 5′ to the central region. The central region varies in length as described elsewhere herein. In an embodiment, splint oligonucleotides possess sequence complementary to both the 5′ and 3′ ends of the cDNA. Splint oligonucleotides anneal to the 5′ and 3′ ends of the cDNA, circularizing the cDNA.

In an aspect of the invention, the reaction mixture comprising cDNA is incubated with a 3′ to 5′ exonuclease. The 3′ to 5′ exonuclease reduces unannealed primer or single-stranded splint oligonucleotides.

In an alternative embodiment, synthesizing cDNA comprises a first strand synthesis step and a second strand synthesis step. In the method, the RNA and primer mixture is incubated with a reverse transcriptase. In an embodiment the nucleotide sequence of the primer is set forth in SEQ ID NO:1 and is phosphorylated at the 5′ terminus. Any available reverse transcriptase, including but not limited to, moloney murine leukemia virus reverse transcriptase, avian myeloblastosis virus reverse transcriptase, recombinant Thermus thermophilus DNA polymerase, and Expand reverse transcriptase may be used in the method of the invention. Reverse transcriptase extends the primer to generate an RNA-cDNA mixture. In an embodiment, the RNA and cDNA mixture is incubated with a ribonuclease H. Second strand synthesis reaction components are incubated with the first strand of cDNA. Second strand synthesis yields double-stranded cDNA. In an embodiment, the RNA and cDNA mixture is incubated with Exonuclease I, which cleaves single-stranded DNA in the 3′ to 5′ direction. In an embodiment, the mixture is incubated with a ribonuclease H and a 3′ to 5′ exonuclease. cDNA may be purified at one or more stages in the methods of the invention.

In an embodiment, a terminal transferase is incubated with linear double-stranded cDNA. Terminal transferase modifies the 3′ termini of cDNA by adding a first homopolymer region. The first homopolymer region is at least 5 homogenous nucleotides. The nucleoside of the first homopolymer region may be deoxyadenosine, deoxyguanosine, deoxycytidine, or thymidine. After the terminal transferase reaction, linear double-stranded cDNA have a first homopolymer region at the 3′ termini of the first and second strands. The first homopolymer regions are single-stranded.

Double-stranded cDNA is incubated with a splint oligonucleotide. The splint oligonucleotide is phosphorylated at the 5′ end. Splint oligonucleotides are isolated nucleic acid molecules comprising a second homopolymer region 3′ to a variable region. In an embodiment, the nucleoside of the second homopolymer region complements the nucleoside of the first homopolymer region. In an embodiment, the second homopolymer region is at least 5 nucleotides. In an embodiment the variable region's nucleotide sequence is a palindrome. In an embodiment, the nucleotide sequence of the variable region includes a restriction enzyme site. In an embodiment, the nucleotide sequence of the splint oligonucleotide is set forth in SEQ ID NO:2. In an embodiment the nucleotide sequence of the splint oligonucleotide is set forth in SEQ ID NO:8. The second homopolymer region of the splint oligonucleotides anneals to the first homopolymer region. The variable regions of the first and second splint oligonucleotides anneal to each other, thus circularizing the cDNA. Alternatively, the variable regions of the first and second splint oligonucleotides anneal to each other, then the second homopolymer regions anneal to the first homopolymer regions on the cDNA. Possible gaps in the annealed molecules are filled in with a DNA polymerase.

The splint molecules anneal to the cDNA ends, bringing the ends in close proximity for ligation. A gap-filling polymerase incorporates bases to fill gaps in the strands. In an embodiment, the cDNA is incubated with a DNA ligase, such as, but not limited to, T4 DNA ligase. In an embodiment, the DNA ligase ligates the circularized cDNA ends to each other. Circular cDNA molecules suitable for rolling circle amplification are formed upon ligation.

The circularized cDNA is incubated with a rolling circle DNA polymerase and rolling circle amplification reaction components. Rolling circle replication amplifies the cDNA. In an embodiment the amplified cDNA is subsequently incubated with a restriction enzyme.

In an aspect of the invention, the amplified cDNA is transcribed into amplified RNA.

In an embodiment, products of the rolling circle amplification are labeled using a variety of methods including, but not limited to, in vitro transcription.

Compositions of the invention include a kit providing reagents for RNA amplification through a method of the invention. A kit of the invention includes, but is not limited to, primer, reverse transcriptase, terminal transferase, a deoxynucleotide triphosphate solution, splint oligonucleotide, ligase, and a rolling circle amplification component. A kit of the invention may also include an RNA isolation component, cDNA synthesis reaction component, ribonuclease H reaction component, 3′ to 5′ exonuclease reaction component, terminal transferase reaction component, cDNA purification component, annealing component, ligase reaction component, a restriction enzyme and a restriction enzyme reaction component, or an in vitro transcription component.

Additional methods of the invention compare RNA expression levels from multiple samples. The methods involve obtaining multiple samples and isolating RNA from each sample. The RNA samples are incubated with primers, and cDNA is synthesized from each sample. The cDNA 3′ termini are modified. The cDNA is incubated with splint oligonucleotides to circularize the double or single-stranded cDNA. The circularized cDNA is sealed, and incubated with rolling circle replication components. The resulting rolling circle replication products from each sample are transcribed into amplified RNA. In an embodiment, the amplified RNA is labeled. Amplified RNA from each sample is incubated with a probe, and the results are analyzed. In an embodiment, the probe is located on a microarray. Alternatively, the probe is in solution.

In an embodiment the invention methods amplify RNA. The methods involve providing RNA; synthesizing cDNA with predetermined nucleotide sequence at the 5′ and 3′ ends of the cDNA from the RNA, circularizing the cDNA, and preparing amplified cDNA from the circularized cDNA using rolling circle replication. In an aspect of the invention the RNA is incubated with a primer. In an embodiment, the primer is an isolated nucleic acid molecule comprising a hairpin region, a promoter region, and a poly(dT) region. In an aspect, the primer is an isolated nucleic acid molecule having a nucleotide sequence comprising the nucleotide sequence set forth in SEQ ID NO:6.

In various aspects of the invention synthesizing cDNA comprises the step of first strand synthesis. Embodiments of the invention comprise incubating the RNA with a reverse transcriptase. Aspects of the invention comprise incubating the cDNA with a 3′ to 5′ exonuclease such as, but not limited to, Exonuclease I. In an embodiment, the method comprises the step of isolating said cDNA.

In aspects of the invention incubating a terminal transferase with the cDNA provides the predetermined nucleotide sequence at the 3′ end of the cDNA. In an embodiment the terminal transferase modifies a 3′ terminus of the cDNA by adding a first homopolymer region. In an aspect the first homopolymer region is at least five homogenous nucleotides. In an aspect, the nucleoside of the first homopolymer region is deoxyadenosine. In an aspect, the method comprises an annealing incubation step. In an embodiment the first homopolymer region anneals with the poly(dT) region. The annealing circularizes the cDNA.

In an embodiment a DNA ligase is incubated with the circularized cDNA prior to amplifying the circularized cDNA. In an aspect amplifying the circularized cDNA using rolling circle replication comprises the step of incubating a rolling circle polymerase with the circularized cDNA. In an aspect the amplified cDNA is incubated with a restriction enzyme prior to transcribing the amplified cDNA. In an embodiment the amplified cDNA is transcribed into amplified RNA.

Embodiments of the invention provide a kit for amplifying RNA. A kit of the invention comprises a primer, reverse transcriptase, terminal transferase, deoxynucleoside triphosphate solution, a ligase, and a rolling circle replication reaction component. In an aspect, the primer is an isolated nucleic acid molecule having the nucleotide sequence set forth in SEQ ID NO:6.

The invention provides methods for comparing RNA expression levels in multiple samples. The methods comprise providing RNA from multiple samples; synthesizing a cDNA with predetermined sequence at the 5′ and 3′ ends of the cDNA from the RNA; circularizing the cDNA; preparing amplified cDNA from the circularized cDNA using rolling circle replication; and evaluating RNA expression levels. In an aspect, the RNA is incubated with a primer comprising a hairpin region. In an aspect the cDNA is incubated with a 3′ to 5′ exonuclease. In an aspect of the method, the method comprises the steps of transcribing the amplified cDNA from each sample into amplified RNA, incubating the amplified RNA with a probe, and analyzing the results.

### BRIEF DESCRIPTION OF THE DRAWINGS

FIG. 1 schematically represents splint oligonucleotide circularization of cDNA. A splint oligonucleotide anneals to the 5′ and 3′ ends of single-stranded cDNA.

FIG. 2 schematically represents splint oligonucleotide circularization of cDNA. Splint oligonucleotides anneal to the 3′ ends of double-stranded cDNA, then the 5′ ends of the splint oligonucleotides anneal.

FIG. 3 schematically represents splint oligonucleotide circularization of cDNA. The 5′ ends of the splint oligonucleotides anneal to each other, then the splint oligonucleotides anneal to the 3′ ends of double-stranded cDNA.

### DETAILED DESCRIPTION OF THE INVENTION

The present invention provides a method for amplification of RNA. Further compositions of the invention include kits that allow one to efficiently amplify RNA. The invention further provides a method for comparison of RNA transcript levels in multiple samples.

By “amplification” is intended an increase in the amount of nucleic acid molecules in a sample. Amplifying RNA or DNA increases the amount of acid precipitable nucleic acid molecules. The amount of acid precipitable material increases by 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, or more. The amount of amplified RNA may increase 100; 1,000; 10,000; 100,000; 1,000,000; 10,000,000; or 100,000,000 fold or more. For instance the methods of the invention may yield 1, 5, 10, 20, 30, 40, 50, 60, 70, 80, 90, 100, 110, 120, 130, 140, 150, 160, 170, 180, 190, 200 or more μg RNA from 100 picograms of total source RNA. The amount of total source RNA used in the methods of the invention is in the range of from 0.001 ng to 1 kg, preferably from 0.001 ng to 1 g, more preferably from 0.001 ng to 1 mg, yet more preferably from 0.001 ng to 1 μg, yet still more preferably from 0.01 ng to 1 μg, yet even still more preferably from 0.1 ng to 1 μg. mRNA may represent 0.1%, 1%, 2%, 3%, 4%, 5%, 6%, 7%, 8%, 9%, or 10% of total source RNA obtained through a method that does not select for mRNA. Methods of quantifying nucleic acid molecules are known in the art and include, but are not limited to, UV absorption spectra, radiolabel incorporation, agarose gel electrophoresis, and ethidium bromide staining. See, for example, Ausubel et al., eds. (2003) Current Protocols in Molecular Biology, (John Wiley & Sons, New York) and Sambrook et al. (2001) Molecular Cloning: A Laboratory Manual (3d ed., Cold Spring Harbor Laboratory Press, Plainview, N.Y., herein incorporated by reference. By “amplified cDNA” is intended the product of rolling circle replication or in vitro amplification. By “amplified RNA” is intended RNA transcribed from amplified cDNA.

The methods of the invention involve isolating RNA and incubating the RNA with a primer. By “isolating” or “purifying” is intended any method that results in isolated or substantially purified nucleic acid molecule. An “isolated” or substantially “purified” nucleic acid molecule, or biologically active portion thereof, is substantially free of other cellular material, or culture medium when produced by recombinant techniques or substantially free of chemical precursors or other chemicals when chemically synthesized. Preferably, 1%, 5%, 10%, 15%, 20%, 25%, 30%, 35%, 40%, 45%, 50%, 55%, 60%, 65%, 70%, 75%, 80%, 85%, 90%, 95%, or more of an isolated nucleic acid molecule sample is the desired nucleic acid molecule. Methods of isolating or purifying RNA and DNA are known in the art and described elsewhere herein. Any method of isolating or purifying RNA or DNA known in the art may be used in the method of the invention.

The methods utilize total RNA or mRNA isolated from one or more samples such as one or more tissues of a subject, experimental subjects and corresponding control subjects, or diseased and healthy cells obtained from one subject. Any RNA isolation technique which does not select against the isolation of mRNA may be utilized for the purification of such RNA samples. Methods of isolating RNA are known in the art. See, for example, Ausubel et al., eds. (2003) Current Protocols in Molecular Biology, (John Wiley & Sons, New York); Sambrook et al. (2001) Molecular Cloning: A Laboratory Manual (3d ed., Cold Spring Harbor Laboratory Press, Plainview, N.Y.; Botwell & Sambrook Eds. (2003) DNA Microarrays (Cold Spring Harbor Laboratory Press, Cold Spring Harbor, N.Y.) which are incorporated herein by reference in their entirety. Additionally, large numbers of tissue samples may readily be processed using techniques well known to those of skill in the art, such as, for example, the single-step RNA isolation process of Chomczynski, P. (U.S. Pat. No. 4,843,155, which is incorporated herein by reference in its entirety).

Methods of isolating RNA are known in the art and include, but are not limited to, chromatography on oligo (dT)-cellulose, biotinylated oligo(dT) and magnetic beads, guanidium thiocyanate removal of cellular contaminants, selective precipitation with ethanol, filter membrane binding, spin columns, vacuum columns, and the methods incorporated in commercially available kits such as the SV Total RNA Isolation procedure (Promega). See, for example, Chirgwin et al (1979) Biochemistry 18:5294-5299; Aviv & Leder (1972) PNAS 69:1408-1412; Ausubel et al., eds. (2003) Current Protocols in Molecular Biology, (John Wiley & Sons, New York) and Sambrook et al. (2001) Molecular Cloning: A Laboratory Manual 3d ed., Cold Spring Harbor Laboratory Press, Plainview, N.Y.; Botwell & Sambrook Eds. (2003) DNA Microarrays (Cold Spring Harbor Laboratory Press, Cold Spring Harbor, N.Y.; herein incorporated by reference in their entirety.

Methods and components for purifying cDNA are known in the art. Any method of purifying cDNA may be used in the methods of the invention. It is recognized that factors such as the preceding step in rolling circle amplification, likely contaminants, and the desired outcome of the subsequent step may determine the most suitable cDNA purification method for a particular stage in rolling circle amplification. It is further recognized that multiple cDNA purification steps may be performed in practicing the methods of the invention.

By “incubating” is intended maintaining environmental conditions favorable to a desired outcome for a period of time. The methods of the invention require incubation of multiple components of the RNA amplification process. The indicated components are combined and incubated. Frequently the incubation includes additional substances that facilitate the desired outcome of the incubation. Incubating RNA amplification process components may be performed under a variety of temperature or reaction conditions. Incubation temperatures may range from 0° C. to 100° C. depending on the components being incubated and the desired outcome of the incubation. Multiple temperatures may be used during the incubation period. Incubation temperatures and conditions for the various components and the desired outcome of the incubations are known in the art. Duration of an incubation may range from 10, 20, 30, 40, 50, to 60 seconds; 1, 2, 3, 4, 5, 6, 7, 8, 9, 10, 15, 20, 25, 30, 35, 40, 45, 50, 55, to 60 minutes; 1, 1.5, 2, 2.5, 3, 3.5, 4, 4.5, 5, 5.5, 6, 7, 8, 9, 10, 11, 12, 24, 36, 48, to 60 hours. Desired outcomes include, but are not limited to, hybridization to RNA, primer extension, RNA degradation, 3′ modification, second strand synthesis, product purification, annealment to cDNA, ligation, cDNA amplification, digestion, transcription, and RNA isolation. Preferred temperatures and conditions for achieving the desired outcomes are discussed elsewhere herein.

By “annealing incubation” is intended an incubation in environmental conditions favorable to annealment of one or more nucleic acid molecules in an intramolecular or intermolecular hybridization.

By “primer” is intended an isolated nucleic acid molecule of defined or random nucleotide sequence. Oligonucleotide primers can be designed to amplify corresponding RNA or DNA sequences from isolated RNA, cDNA or genomic DNA extracted from any organism of interest. Typically primers are short in sequence. In an embodiment, a primer is less than about 100, 90, 80, 70, 60, 55, 50, 45, 40, 35, 30, 25, 20, 15, 10, or 5 nucleotides in length. Methods for designing primers are generally known in the art and are disclosed in Sambrook et al. (1989) Molecular Cloning: A Laboratory Manual (2d ed., Cold Spring Harbor Laboratory Press, Plainview, N.Y.; Botwell & Sambrook Eds. (2003) DNA Microarrays (Cold Spring Harbor Laboratory Press, Cold Spring Harbor, N.Y.)). Primers of the invention comprise the nucleotide sequences set forth in SEQ ID NO:1, SEQ ID NO:6, and SEQ ID NO:7.

In the methods of the invention, primers comprise a poly(dT) region. In an embodiment, a primer comprises a 5′ phosphorylated poly(dt) region. The poly(dT) region is a nucleotide sequence of at least 5, 6, 7, 8, 9, 10, 12, 15, 18, 20, 21, 24, 27, 30, or more contiguous thymidine bases. In embodiments of the invention primers comprise a promoter region, a middle region, and a poly(dT) region, arranged in 5′ to 3′ order. Promoter regions are nucleotide sequences capable of initiating transcription. Promoter regions include, but are not limited to the T7 promoter region set forth in SEQ ID NO:3, the SP6 promoter region set forth in SEQ ID NO:4, and the T3 promoter set forth in SEQ ID NO:5, and fragments or variants thereof that are capable of initiating transcription. The middle region of the primer is the nucleotide sequence between the nucleotide sequences necessary for initiating transcription in the promoter region and the poly(dT) region. The middle region varies in length and nucleotide sequence composition from 0 to 1, 2, 3, 4, 5, 6, 7, 8, 9, 10, 15, 20, 25, 30, 35, 40, 45, 50 or more nucleotides.

Fragments and variants of the nucleotide sequences of interest are also encompassed by the present invention. By “fragment” is intended a portion of the nucleotide sequence. By “variants” is intended substantially similar sequences. Naturally occurring allelic variants such as can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis. Generally, variants of a particular nucleotide sequence of interest will have at least about 90%, 91%, 92%, 93%, 94%, 95%, 96%, 97%, and more preferably at least about 98%, 99% or more sequence identity to that particular nucleotide sequence as determined by sequence alignment programs described elsewhere herein using default parameters.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

(b) As used herein “comparison window” makes reference to a contiguous and specified segment of a polynucleotide sequence, wherein the polynucleotide sequence in the comparison window may comprise additions or deletions (i.e. gaps) compared to the reference sequence (which does not comprise additions or deletions) for optimal alignment of the two sequences. Generally the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Preferred, non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. For purposes of the present invention, comparison of nucleotide or protein sequences for determination of percent sequence identity to the sequences disclosed herein is preferably made using the GCG program GAP (Version 10.00 or later) with its default parameters or any equivalent program. By “equivalent program” is intended any sequence comparison program that, for any two sequences in question, generates an alignment having identical nucleotide matches and an identical percent sequence identity when compared to the corresponding alignment generated by the preferred program.

Sequence comparison programs include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM 120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nln.nih.gov. Alignment may also be performed manually by inspection.

(c) As used herein, “sequence identity” or “identity” in the context of two nucleic acid sequences makes reference to the residues in the two sequences that are the same when aligned for maximum correspondence over a specified comparison window.

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters.

Another indication that nucleotide sequences are substantially complementary is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein.

In various embodiments, a primer of the invention further comprises a hairpin region. In an embodiment the nucleotide sequence of a hairpin primer is set forth in SEQ ID NO:6. Hairpin regions comprise a single-stranded loop region and a duplex stem region. Single-stranded loop regions vary in length from 1 nucleotide to 100 nucleotides, preferably from 2 nucleotides to 50 nucleotides, more preferably from 3 nucleotides to 25 nucleotides, yet more preferably from 3 nucleotides to 15 nucleotides. Duplex stem regions vary in length from 1 nucleotide to 500 nucleotides, preferably from 1 nucleotide to 100 nucleotides, more preferably from 2 nucleotides to 50 nucleotides, yet more preferably from 2 nucleotides to 25 nucleotides. It is acknowledged that the duplex region of the hairpin may extend into other regions of the primer such as the promoter region.

In an embodiment, the poly(dT) region of the primer anneals to the polyA tail on mRNA molecules. Hybridization of the poly(dT) region to the polyA tail on mRNA provides a primer for first strand cDNA synthesis. In an embodiment, a primer is annealed to RNA yielding an “RNA and primer mixture.”

Annealing of nucleotide molecules is known in the art. Methods and components for optimizing annealing or hybridizing are described elsewhere herein and in Ausubel et al., eds. (2003) Current Protocols in Molecular Biology, (John Wiley & Sons, New York); Sambrook et al. (2001) Molecular Cloning: A Laboratory Manual (3d ed., Cold Spring Harbor Laboratory Press, Plainview, N.Y.; Botwell & Sambrook Eds. (2003) DNA Microarrays (Cold Spring Harbor Laboratory Press, Cold Spring Harbor, N.Y.), herein incorporated by reference in their entirety.

By “anneal” is intended the pairing of complementary DNA or RNA sequences, via hydrogen bonding, to form a double-stranded molecule. Annealing is often used to describe the binding of a short primer or probe. Methods for determining preferred incubation temperatures when annealing or hybridizing nucleic acid molecules are desired outcomes are known in the art. Suitable incubation temperatures for annealing a primer to RNA or a splint oligonucleotide to cDNA can be determined based on the primer or oligonucleotide sequence. Methods for determining annealing temperatures are generally known in the art and are disclosed in Sambrook et al. (1989) Molecular Cloning: A Laboratory Manual (2d ed., Cold Spring Harbor Laboratory Press, Plainview, N.Y.).

For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched nucleic acid molecule. Tₘ is reduced by about 1° C. for each 1% of mismatching; thus, Tₘ, hybridization, and/or wash conditions can be adjusted to hybridize to sequences of the desired identity. For example, if annealing of sequences with approximately 90% identity is sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); Ausubel et al., eds. (2003) Current Protocols in Molecular Biology, (John Wiley & Sons, New York); and Sambrook et al. (2001) Molecular Cloning: A Laboratory Manual (3d ed., Cold Spring Harbor Laboratory Press, Plainview, N.Y., herein incorporated by reference in their entirety

By “synthesizing cDNA” is intended the process of generating single-stranded or double-stranded DNA of which one strand complements a RNA strand. Synthesizing cDNA is a procedure known in the art and involves the use of primers and reverse transcriptases for first strand synthesis. As used herein “cDNA synthesis” refers to first strand synthesis or first and second strand synthesis. Both first strand synthesis and first and second strand synthesis reactions yield cDNA. cDNA synthesis reactions are known in the art. cDNA synthesis reaction components include but are not limited to first and second strand synthesis components. See for example, Ausubel et al., eds. (2003) Current Protocols in Molecular Biology, (John Wiley & Sons, New York) and Sambrook et al. (2001) Molecular Cloning: A Laboratory Manual (3d ed., Cold Spring Harbor Laboratory Press, Plainview, N.Y.).

In an embodiment, synthesizing cDNA comprises a first strand synthesis step. In the method, the RNA and primer mixture is incubated with a reverse transcriptase. A “reverse transcriptase” is an enzyme capable of utilizing an RNA molecule as a template and polymerizing a complementary DNA molecule. The reverse transcriptase requires a primer bound to RNA to initiate the polymerization reaction. Reverse transcriptases are known in the art and include, but are not limited to, moloney murine leukemia virus reverse transcriptase, avian myeloblastosis virus reverse transcriptase, recombinant Thermus thermophilus DNA polymerase, Superscript™ II and Expand™ reverse transcriptase. A reverse transcriptase is incubated with the RNA and primer mixture in reaction conditions suitable to the particular reverse transcriptase and the primer. Reverse transcriptase extends the primer to generate an RNA-cDNA mixture. By “extends” is intended polymerizes nucleoside triphosphates onto a nucleic acid molecule such as a primer or DNA strand. The primer introduces predetermined nucleotide sequence at the 5′ ends of the cDNA.

By “first strand synthesis” is intended the polymerization of a cDNA strand on a strand of RNA by a reverse transcriptase. The product of first strand synthesis is a double-stranded molecule comprising a DNA strand and an RNA strand. The first cDNA strand complements the sequence of the RNA strand. See Krug & Berger (1987) Meth. Enzymol. 152:316-325, herein incorporated by reference in its entirety.

By “reaction component” is intended any substance that facilitates the indicated reaction. Reaction components that facilitate the reaction may or may not participate in the chemical processes of the reaction. Reaction components include, but are not limited to, vessels, such as microfuge tubes and multiwell plates; measuring devices, such as micropipette tips and capillary tubes; filters; separation devices such as microfuge tube filter inserts, vacuum apparati, purification resins, magnetic beads, and columns; reagents; compounds; solutions; molecules; buffers; inhibitors; chelating agents; ions; terminators; stabilizers; precipitants; solubilizers; acids; bases; salts; reducing agents; oxidizing agents; enzymes; catalysts; and denaturants. In an embodiment of the invention, concentrated reaction components are provided in kits of the invention. The concentration of the reaction components provided in a kit of the invention may be 1, 2, 3, 4, 5, 6, 7, 8, 9, 10, 15, 20, 25, 30, 35, 40, 50, 60, 70, 80, 90, 100, or more fold concentrated than the desired concentration of the component in the reaction. Reagents and reaction level concentrations of various reagents are discussed elsewhere herein.

Typical first strand synthesis reaction components include, but are not limited to, sodium pyrophosphate, Tris-HCl, KCl, MgCl₂, MnCl₂, spermidine, dithiothreitol, dATP, dCTP, dGTP, and dTTP. The sodium pyrophosphate concentration in a reverse transcriptase reaction may range from 0 to 100 mM, preferably 0.01 to 70, more preferably 0.1 to 50, yet more preferably from 1 to 20. Such concentrations include but are not limited to 0.01, 0.1, 0.5, 1, 2, 3, 4, 5, 6, 7, 8, 9, 10, 11, 12, 13, 14, 15, 16, 17, 18, 19, 20, 30, 40, 50, 60, 70, 80, 90 and 100 mM sodium pyrophosphate. The Tris-HCl pH in a reverse transcriptase reaction may range from pH 7.2 to 9.2, preferably 7.5 to 8.8. The pH in a typical reverse transcriptase reaction may be a pH of 7.2, 7.5, 7.8, 8.0, 8.3, 8.5, 8.8, 9.0, or 9.2. The Tris-HCl concentration in a reverse transcriptase reaction may range from 0 to 250 mM, preferably 0.01 to 125, more preferably 0.1 to 50, yet more preferably from 1 to 25. Such concentrations include but are not limited to 0, 0.5, 1, 10, 20, 30, 40, 50, 60, 70, 80, 90, 100, 125, 150, 200, and 250 mM Tris-HCl. The KCl concentration in a reverse transcriptase reaction may range from 0 to 250 mM, preferably 0.01 to 125, more preferably 0.1 to 62.5, yet more preferably from 1 to 25. Such concentrations include but are not limited to 0, 0.1, 1, 2, 3, 4, 5, 6, 7, 8, 9, 10, 20, 30, 40, 50, 60, 70, 75, 80, 85, 90, 95, 100, 120, 140, 160, 180, 200, 220, or 250 mM KCl. The MgCl₂ concentration in a reverse transcriptase reaction may range from 0 to 100 mM, preferably 0.01 to 62.5, more preferably 0.1 to 20, yet more preferably from 1 to 10. Such concentrations include but are not limited to 0.01, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, to 100 mM MgCl₂. The MnCl₂ concentration in a reverse transcriptase reaction may range from 0 to 100 mM, preferably from 0.1 to 50 mM, more preferably from 1 to 10 mM. Such concentrations include, but are not limited to, 0.01, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, and 100 mM MnCl₂. The spermidine concentration in a reverse transcriptase reaction may range from 0 to 5 mM, preferably 0.01 to 2 mM, more preferably 0.1 to 1 mM. Such concentrations include, but are not limited to 0, 0.01, 0.05, 0.1, 0.2, 0.3, 0.4, 0.5, 0.6, 0.7, 0.8, 0.9, 1, 2, 3, 4, and 5 mM spermidine. The dithiothreitol concentration in a reverse transcriptase reaction may range from 0 to 100 mM, preferably 0.1 to 20 mM, more preferably from 0.1 to 1 mM. Such concentrations include, but are not limited to, 0, 0.1, 1, 2, 3, 4, 5, 6, 7, 8, 9, 10, 11, 12, 13, 14, 15, 16, 17, 18, 19, 20, 25, 30, 40, 50, 60, 70, 80, 90, and 100 mM dithiothreitol. The β-mercaptoethanol concentration in a reverse transcriptase reaction may range from 0 to 100 mM, preferably 0.1 to 20 mM, more preferably from 0.1 to 1 mM. Such concentrations include, but are not limited to, 0, 0.1, 1, 2, 3, 4, 5, 6, 7, 8, 9, 10, 11, 12, 13, 14, 15, 16, 17, 18, 19, 20, 25, 30, 40, 50, 60, 70, 80, 90, and 100 mM β-mercaptoethanol. The dATP concentration in a reverse transcriptase reaction may range from 0.01 to 20 mM, preferably 0.1 to 10 mM, more preferably 1 to 10 mM. Such concentrations include, but are not limited to, 0.01, 0.05, 0.1, 0.5, 1, 2, 3, 4, 5, 6, 7, 8, 9, 10, 15, to 20 mM dATP. The dCTP concentration in a reverse transcriptase reaction may range from 0.01 to 20 mM, preferably 0.1 to 10 mM, more preferably 1 to 10 mM. Such concentrations include, but are not limited to, 0.01, 0.05, 0.1, 0.5, 1, 2, 3, 4, 5, 6, 7, 8, 9, 10, 15, to 20 mM dCTP. The dGTP concentration in a reverse transcriptase reaction may range from 0.01 to 20 mM, preferably 0.1 to 10 mM, more preferably 1 to 10 mM. Such concentrations include, but are not limited to, 0.01, 0.05, 0.1, 0.5, 1, 2, 3, 4, 5, 6, 7, 8, 9, 10, 15, to 20 mM dGTP. The dTTP concentration in a reverse transcriptase reaction may range from 0.01 to 20 mM, preferably 0.1 to 10 mM, more preferably 1 to 10 mM. Such concentrations include, but are not limited to, 0.01, 0.05, 0.1, 0.5, 1, 2, 3, 4, 5, 6, 7, 8, 9, 10, 15, to 20 mM dTTP.

Reaction conditions for the various reverse transcriptases are known in the art. Reverse transcription reaction incubations include, but are not limited to, incubations at temperatures ranging from 30° C., 33° C., 35° C., 37° C., 40° C., 42° C., 45° C., 50° C., 55° C., 60° C., 65° C., 70° C., 75° C., 80° C., to 85° C. Durations of incubation periods for reverse transcription reactions range from 1, 5, 10, 20, 30, 40, 50, 60, 70, 80, 90, 100, 110, and 120 minutes or more. Factors to consider in determining the desired incubation temperature include the reverse transcriptase and the duration of the reaction. See, for example, Ausubel et al., eds. (2003) Current Protocols in Molecular Biology, (John Wiley & Sons, New York) and Sambrook et al. (2001) Molecular Cloning: A Laboratory Manual (3d ed., Cold Spring Harbor Laboratory Press, Plainview, N.Y., herein incorporated by reference.

By “ribonuclease H” or “RNase H” is intended an endoribonuclease that specifically degrades the RNA strand of an RNA-DNA hybrid to produce 5′ phosphate-terminated and 3′-hydroxyl terminated oligoribonucleotides and single-stranded DNA. Ribonuclease H's are known in the art, and include, but are not limited to, E. coli RNase H, Avian Myeloblastosis Virus reverse transcriptase, and Moloney Murine Leukemia Virus reverse transcriptase.

RNase H reaction components are known in the art and include, but are not limited to, Hepes-KOH, KCl, MgCl₂, and DTT. The Hepes-KOH concentration in a ribonuclease H reaction may range from 0 to 200 mM, preferably 0.2 to 20 mM, more preferably 1 to 10 mM. Such concentrations include, but are not limited to, 0, 0.2, 2, 20, to 200 mM Hepes-KOH. The Hepes-KOH pH in a ribonuclease H reaction may range from 7.0 to 9.6, preferably 7.3 to 9.0, more preferably 7.3 to 8.5. Such pHs include, but are not limited to, 7.0, 7.1, 7.2, 7.3, 7.4, 7.5, 7.6, 7.7, 7.8, 8.0, 8.1, 8.2, 8.3, 8.4, 8.5, 8.6, 8.7, 8.8, 8.9, 9.0, 9.1, 9.2, 9.3, 9.4, 9.5 and 9.6. The KCl concentration in a ribonuclease H reaction may range from 0 to 250 mM, preferably 0.01 to 125, more preferably 0.1 to 62.5, yet more preferably from 1 to 25. Such concentrations include but are not limited to 0, 0.1, 1, 2, 3, 4, 5, 6, 7, 8, 9, 10, 20, 30, 40, 50, 60, 70, 75, 80, 85, 90, 95, 100, 120, 140, 160, 180, 200, 220, or 250 mM KCl. The MgCl₂ concentration in a reverse transcriptase reaction may range from 0 to 100 mM, preferably 0.01 to 62.5, more preferably 0.1 to 20, yet more preferably from 1 to 10. Such concentrations include but are not limited to 0.01, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, to 100 mM MgCl₂. The dithiothreitol concentration in a ribonuclease H reaction may range from 0 to 10 mM, preferably 0.01 to 10 mM, more preferably 1 to 10. Such concentrations include, but are not limited to, 0, 0.01, 0.1, 1, to 10 mM DTT.

Conditions for ribonuclease H reactions are known in the art. Ribonuclease H reaction incubations include, but are not limited to, incubations at temperatures ranging from 15° C., 25° C., 30° C., 35° C., 37° C., 40° C., 42° C., 45° C., 50° C., to 55° C. Durations of incubation periods for ribonuclease H reactions range from 0, 2, 4, 6, 8, 10, 15, 20, 25, 30, 35, 40, 45, 50, 55, 60, 65, 70, 75, 80, 85, to 90 minutes or more. The ribonuclease H used in the reaction is a factor that indicates suitable incubation temperatures and durations. See, for example, Ausubel et al., eds. (2003) Current Protocols in Molecular Biology, (John Wiley & Sons, New York) and Sambrook et al. (2001) Molecular Cloning: A Laboratory Manual (3d ed., Cold Spring Harbor Laboratory Press, Plainview, N.Y., herein incorporated by reference.

In an embodiment, the methods of the invention generate linear single-stranded DNA with defined sequence at the 5′ and 3′ ends. The linear cDNA produced by first strand synthesis is incubated with terminal transferase. Terminal transferase modifies a 3′ terminus of the cDNA by adding a first homopolymer region. The first homopolymer region is at least 5 homogenous nucleotides. In an embodiment the homogenous nucleotides are consecutive. In an embodiment, the homogenous nucleotides are interspersed with inosine. The nucleoside of the first homopolymer region may be deoxyadenosine, deoxyguanosine, deoxycytidine, or thymidine. In the jargon of molecular biology, nucleotide and base are often used to refer to a nucleoside. In an embodiment, the first homopolymer region is adenosine. In an embodiment, the first homopolymer region is deoxycytidine. After the terminal transferase reaction, the linear single-stranded cDNAs have defined sequence at both the 5′ and 3′ ends. Homopolymer regions can be introduced to the cDNA through a variety of methods, including but not limited to, incubation with a terminal transferase or an oligonucleotide such as a splint oligonucleotide. In an embodiment of the invention the bases of the first and second homopolymer regions complement each other. The number of homogenous bases in a homopolymer region ranges from 5, 10, 15, 20, 30, 40, 50, 60, 70, 80, 90, 100, 200, 300, 400, 500, 600, 700, 800, 900, to 1000 or more bases.

By “modifying” is intended changing or altering. Modifying a molecule includes, but is not limited to, extending, phosphorylating, dephosphorylating, linking, polymerizing, joining, ligating, degrading, and cleaving the molecule.

By “terminal transferase” or “terminal deoxynucleotidyl transferase” is intended a template independent polymerase that catalyzes the repetitive addition of deoxynucleotides to the 3′ hydroxyl terminus of DNA molecules with the concomitant release of inorganic phosphate. The incorporation efficiency for the four nucleotides varies; thus the nucleotide present in the reaction affects the number of nucleotides incorporated in an incubation period. Terminal transferases are known in the art and are described in Chang et al. (1986) Crit. Rev. Biochem. 21:27-52 and Roychoudhury et al. (1976) Nucleic Acids Research. 3:101-116, herein incorporated by reference in their entirety.

Terminal transferase reaction components include, but are not limited to, potassium acetate, Tris-acetate, magnesium acetate, dithiothreitol, cacodylic acid potassium cacodylate, Tris-HCl, acetylated bovine serum albumin (BSA), and CoCl₂. The potassium acetate concentration in a terminal transferase reaction ranges from 0 to 250 mM, preferably 0.5 to 100 mM, more preferably 1 to 50 mM. Such concentrations include, but are not limited to, 0, 0.5, 1, 5, 10, 50, 100 to 250 mM. The Tris-acetate concentration in a terminal transferase reaction ranges from 0 to 200 mM, preferably 0.02 to 100 mM, more preferably 0.2 to 20 mM. Such concentrations include, but are not limited to, 0, 0.02, 0.2, 1, 2, 10, 20, 30, 40, 50, 60, 70, 80, 90, 100, 110, 120, 130, 140, 150, 160, 170, 180, 190, to 200 mM. The magnesium acetate concentration in a terminal transferase reaction ranges from 0 to 100 mM, preferably from 0.01 to 10, more preferably from 0.1 to 10 mM. Such concentrations include, but are not limited to, 0, 0.01, 0.1, 1, 10, to 100 mM. The dithiothreitol concentration in a terminal transferase reaction ranges from 0 to 100 mM, preferably 0.1 to 20 mM, more preferably from 0.1 to 1 mM. Such concentrations include, but are not limited to, 0, 0.001, 0.01, 0.1, 1, 10, to 100 mM. The cacodylic acid concentration in a terminal transferase reaction ranges from 0 to 500 mM, preferably 1 to 400 mM, more preferably 10 to 300 mM. Such concentrations include, but are not limited to, 0, 1, 10, 100, 200, 300, 400, to 500 mM. The potassium cacodylate concentration in a terminal transferase reaction ranges from 0 to 1000 mM, preferably 2 to 500 mM, more preferably from 2 to 50. Such concentrations include, but are not limited to, 0, 2, 10, 20, 50, 100, 200, 300, 400, 500, 600, 700, 800, 900 to 1000 mM. The Tris-HCl concentration in a terminal transferase reaction ranges from 0 to 100 mM, preferably 0.1 to 75 mM, more preferably from 1 to 50. Such concentrations include, but are not limited to, 0, 0.1, 0.2, 0.5, 1, 2, 5, 10, 20, 25, 30, 35, 40, 45, 55, 60, 65, 70, 75, 80, 85, 90, 95, to 100 mM. The BSA concentration in a terminal transferase reaction ranges from 0 to 1000 μg/ml, preferably from 2.5 to 500 μg/ml, more preferably from 10 to 100 μg/ml. Such concentrations include, but are not limited to, 0, 2.5, 25, 250, 500, 750, to 1000 μg/ml. The CoCl₂ concentration in a terminal transferase reaction ranges from 0 to 25 mM, preferably 0.01 to 10, more preferably from 0.1 to 5 mM. Such concentrations include, but are not limited to, 0, 0.01, 0.025, 0.1, 0.25, 0.5, 1, 2.5, 5, 10, to 25 mM. The pH of a terminal transferase reaction ranges from pH 6.0 to 9.0, preferably 6.6 to 8.4, more preferably from 7.0 to 8.0. Such pHs include, but are not limited to, pH 6, 6.2, 6.4, 6.6, 6.8, 7.0, 7.2, 7.4, 7.6, 7.8, 8.0, 8.2, 8.4, 8.6, 8.8 to 9.0.

Terminal transferase reaction conditions are known in the art. Reaction incubations include, but are not limited to incubations at temperatures ranging from 15° C., 20° C., 25° C., 30° C., 35° C., 37° C., 40° C., 42° C., 45° C., 50° C., to 55° C. Incubation period durations for terminal transferase reactions range from 0, 0.5, 1, 5, 10, 15, 20, 25, 30, 35, 40, 45, 50, 55, 60, 65, 70, 75, 80, 85, to 90 minutes or more. Factors that affect the reaction temperature and duration include, but are not limited to, the terminal transferase and the nucleotide triphosphate. See, for example, Ausubel et al., eds. (2003) Current Protocols in Molecular Biology, (John Wiley & Sons, New York) and Sambrook et al. (2001) Molecular Cloning: A Laboratory Manual (3d ed., Cold Spring Harbor Laboratory Press, Plainview, N.Y., herein incorporated by reference.

In an embodiment, the linear single-stranded cDNA with defined sequence at the 5′ and 3′ ends is incubated with a splint oligonucleotide. By “splint oligonucleotide” is intended an isolated nucleic acid molecule comprising a homopolymer region and a variable region. The homopolymer region of the splint oligonucleotide is 3′ to the variable region. By “variable region” is intended a predetermined nucleotide sequence. The predetermined nucleotide sequence of the variable region may differ between any two applications of the methods of the invention. In an embodiment the nucleotide sequence of the variable region comprises a palindrome. In one embodiment of the invention the nucleotide sequence of the variable region comprises a restriction enzyme site. In an embodiment the nucleotide sequence of the variable region comprises a primer complementary region and a central region. By “primer complementary region” is intended a nucleotide sequence that complements at least the 5 5′ terminal nucleotides of the nucleotide sequence of the primer initially annealed to the isolated RNA. The primer complementary region's nucleotide sequence complements 5, 10, 15, 20, 25, 30 nucleotides, or up to the total number of nucleotides in the primer. By “central region” is intended the nucleotide sequence located between the primer complementary region and the polynucleotide region of the splint oligonucleotide. The length of the central region varies from 0 to 1, 2, 3, 4, 5, 6, 7, 8, 9, 10, 15, 20, 25, 30, 35, 40, 45, 50 or more nucleotides. In an embodiment, the central region comprises a restriction enzyme site. In an aspect of the invention, the central region comprises a promoter region (described elsewhere herein).

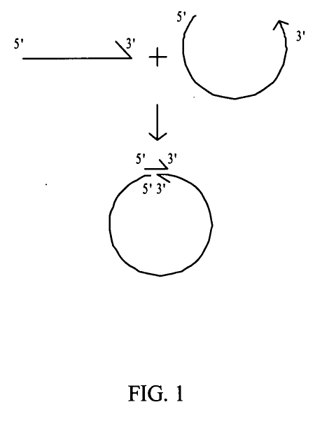

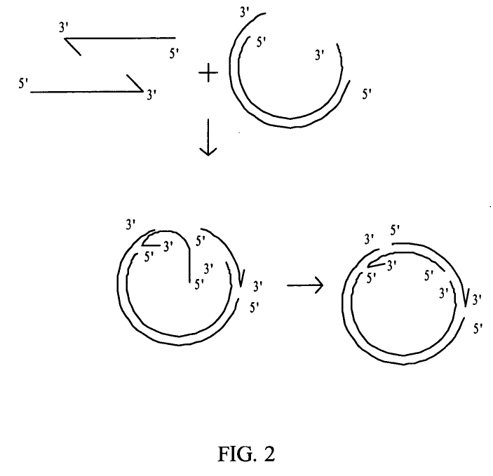

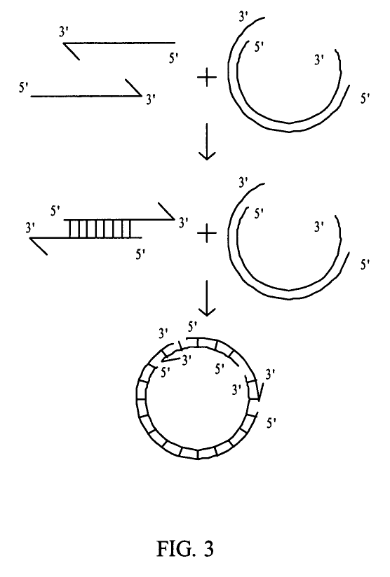

Splint oligonucleotide annealment brings the 5′ and 3′ ends of the cDNA into proximity with each other and stabilizes the ends near each other. By “circularizing” is intended joining the ends of linear cDNA resulting in molecules without free ends. The process of circularizing cDNA involves bringing the 5′ and 3′ ends of the cDNA in proximity to each other, stabilizing the ends near each other, filling in gaps between the ends with nucleotides, and forming phosphodiester bonds between the 5′ and 3′ ends. In an embodiment the splint oligonucleotide anneals to the 5′ and 3′ ends of single-stranded cDNA resulting in a molecule such as the molecule depicted in FIG. 1. In an embodiment splint oligonucleotides anneal to the 3′ ends of double-stranded cDNA, and the splint oligonucleotides anneal to each other (FIG. 2). In an embodiment, splint oligonucleotides anneal to each other forming a double-stranded molecule with 3′ single-stranded regions. The 3′ single-stranded regions anneal to the 3′ ends of double-stranded cDNA (FIG. 3). In an embodiment, splint oligonucleotides link the 5′ and 3′ ends of multiple cDNA molecules. In an embodiment, the splint oligonucleotides and linear cDNA anneal in a combination of the above described annealing processes. It is envisioned that one or more splint oligonucleotides anneal to the cDNA ends.

The splint molecules anneal to the cDNA ends, bringing the ends in close proximity for ligation. A gap-filling polymerase incorporates bases to fill-in gaps in the strands. In an embodiment, the cDNA is incubated with a DNA ligase, such as, but not limited to, T4 DNA ligase. In an embodiment, the DNA ligase ligates the circularized cDNA ends to each other. Circular cDNA molecules suitable for rolling circle amplification are formed upon ligation.

A gap between the cDNA ends will not exceed twice the number of nucleotides in the splint oligonucleotide. The splint oligonucleotide serves as a template for filling in gaps between the ends of the cDNA. Methods of filling in gaps are known in the art and include the use of template-dependent polymerases such as the Klenow fragment of E. coli polymerase I. By “gap filling polymerase” is intended a DNA polymerase capable of template-dependent repair of DNA gaps. Such gap-filling polymerases include, but are not limited to, the Klenow fragment of E. coli polymerase I. See, for example, Ausubel et al., eds. (2003) Current Protocols in Molecular Biology, (John Wiley & Sons, New York) and Sambrook et al. (2001) Molecular Cloning: A Laboratory Manual (3d ed., Cold Spring Harbor Laboratory Press, Plainview, N.Y., herein incorporated by reference.

By “DNA ligase” is intended an enzyme that catalyzes the formation of a phosphodiester bond between juxtaposed 5′-phosphate and 3′-hydroxyl termini in duplex DNA containing cohesive ends. By “ligates” is intended catalyzes the formation of a phosphodiester bond between juxtaposed 5′-phosphate and 3′-hydroxyl termini in duplex DNA containing cohesive ends. DNA ligases are known in the art and include, but are not limited to, T4 DNA ligase, Quick DNA ligase, E. coli ligase, and Tsc DNA ligase.

Typical ligase reaction components include, but are not limited to Tris-HCl, KCl, MgCl₂, Nonidet P40, NAD, DTT, ATP, BSA, and polyethylene glycol 6000. The Tris-HCl concentration in a ligase reaction ranges from 0 to 700 mM, preferably 0.2 to 200 mM, more preferably from 1 to 100 mM. Such concentrations include, but are not limited to, 0, 0.2, 0.3, 0.5, 0.6, 1, 2, 3, 5, 6, 10, 20, 30, 40, 50, 60, 66, 70, 80, 90, 100, 200, 300, 400, 500, 600, and 700 mM. The Tris-HCl pH in a ligase reaction may range from about 7.0 to 8.3, preferably 7.0 to 8.0, more preferably from 7.3 to 7.7. Such pHs include, but are not limited to, 7.0, 7.2, 7.3, 7.4, 7.5, 7.6, 7.7, 7.8, 7.9, 8.0, 8.1, 8.2, and 8.3. The KCl concentration in a ligation reaction may range from 0 to 200 mM, preferably 0.5 to 100 mM, more preferably 5 to 50 mM. Such concentrations include, but are not limited to, 0, 0.5, 1, 5, 10, 15, 20, 25, 30, 35, 40, 45 50, 60, 70, 80, 90, 100, and 200 mM KCl. The MgCl₂ concentration in a ligation reaction may range from 0 to 100, preferably from 0.01 to 50, more preferably from 0.04 to 20 mM. Such concentrations include, but are not limited to, 0, 0.01, 0.04, 0.1, 0.4, 1, 4, 10, 20, 30, 40, 50, and 100 mM. The Nonidet P40 concentration in a ligation reaction may range from 0 to 5%, preferably from 0.01% to 1%, more preferably from 0.1% to 1%. Such concentrations include, but are not limited to, 0, 0.01%, 0.1%, 1%, to 5%. The NAD concentration in a ligation reaction may range from 0 to 2000 μM, preferably from 0.25 to 1000 μM, more preferably from 1 to 250 μM. Such concentrations include, but are not limited to, 0, 0.25, 0.5, 1, 2.5, 5, 10, 25, 26, 50, 100, 250, 500, 750, 1000, 1100, 1200, 1300, 1400, 1500, 1600, 1700, 1800, 1900, and 2000 μM. The DTT concentration in a ligation reaction may range from 0 to 100 mM, preferably from 0.01 to 50 mM, more preferably from 0.1 to 10 mM. Such concentrations include, but are not limited to, 0, 0.01, 0.1, 1, 10, 20, 30, 40, 50, 60, 70, 80, 90 to 100 mM DTT. The ATP concentration in a ligation reaction may range from 0 to 30, preferably from 0.01 to 10, more preferably from 0.1 to 5 mM ATP. Such concentrations include, but are not limited to, 0, 0.01, 0.1, 1, 10, 20, to 30 mM. The BSA concentration in a ligation reaction may range from 0 to 100 μg/ml, preferably from 0.5 to 50 μg/ml, more preferably from 1 to 20 μg/ml. Such concentrations include, but are not limited to, 0, 0.5, 1, 5, 10, 20, 30, 40, 50, 60, 70, 80, 90, to 100 μg/ml. The polyethylene glycol 6000 concentration in a ligation reaction may range from 0 to 20%, preferably 0.1 to 12.5%, more preferably from 1 to 7.5%. Such concentrations include, but are not limited to, 0, 0.1, 0.25, 0.5, 0.75, 1, 2.5, 5, 7.5, 10, 12.5, 15, to 20%.

Ligation reaction conditions are known in the art. Ligation incubations include, but are not limited to, incubations at temperatures ranging from 4° C., 6° C., 8° C., 10° C., 12° C., 15° C., 16° C., 18° C., 20° C., 22° C., 25° C., 30° C., 35° C., 40° C. 45° C., 50° C., 55° C., 60° C., 65° C., 70° C., to 75° C. Incubation period durations for ligations range from 0.5, 1, 5, 10, 15, 20, 25, 30, 35, 40, 45, 50, 55, 60, 65, 70, 75, 80, 85, 90 minutes; 2, 3, 4, 5, 6, 7, 8, 9, 10, 11, 12, 13, 14, 15, 16, 17, 18, 19, 20, 21, 22, 23, to 24 hours or more. The type of ligase utilized is a factor in determining suitable incubation temperatures and durations. See, for example, Ausubel et al., eds. (2003) Current Protocols in Molecular Biology, (John Wiley & Sons, New York) and Sambrook et al. (2001) Molecular Cloning: A Laboratory Manual (3d ed., Cold Spring Harbor Laboratory Press, Plainview, N.Y., herein incorporated by reference.

Prior to rolling circle amplification, the reaction components are incubated with a 3′ to 5′ exonuclease. By “3′ to 5′ exonuclease” is intended an enzyme that is capable of cleaving nucleotides sequentially from the free 3′ end of a linear nucleic acid molecule. The linear nucleic acid molecule may be double-stranded or single-stranded. Preferably, the exonuclease is single-strand specific. A single-strand specific exonuclease, such as Exonuclease I, reduces excess primer or splint oligonucleotide which may interfere with subsequent reaction steps.

The circularized cDNA is incubated with a rolling circle DNA polymerase and rolling circle amplification reaction components. Rolling circle replication amplifies the cDNA. By “rolling circle replication,” “rolling circle amplification,” or “σ-replication” is intended a mode of DNA replication that involves processive, template-dependent polymerization of DNA molecules on a circular DNA template. Rolling circle replication is known in the art and described in Freifelder, D. (1987) Molecular Biology. Jones & Bartlett Publishers, Inc.; Chastain et al (2003). J. Biol. Chem. 278:21276-21285; Gilbert et al (1968) Cold Spring Harb Symp Quant Biol. 33:473-84; Khan 2000 Mol. Microbiol 37:477-484; herein incorporated by reference in their entirety. Rolling circle amplification is highly efficient, with 1000 to 1,000,000 fold amplification per cycle achievable.

By “rolling circle DNA polymerase” is intended a DNA dependent, processive, strand-displacing polymerase. Rolling circle DNA polymerases are known in the art and include, but are not limited to, Φ29 DNA polymerase, TempliPhi, and Φ129. See for example, Lewin, B. 2000 Genes VII Oxford University Press, Oxford, England; Nelson et al. (2002) Biotechniques June Suppl 44-7; Voisey et al (2001) Biotechniques 31:1122-1124; herein incorporated by reference in their entirety.

Rolling circle amplification reactions are known in the art. Protocols and reagents for rolling circle amplification reactions are readily available to the practitioner. See, for example, Ausubel et al., eds. (2003) Current Protocols in Molecular Biology, (John Wiley & Sons, New York) and Sambrook et al. (2001) Molecular Cloning: A Laboratory Manual (3d ed., Cold Spring Harbor Laboratory Press, Plainview, N.Y., herein incorporated by reference.

In an embodiment, the amplified cDNA is incubated with a restriction enzyme. By “restriction enzyme” is intended an enzyme that cleaves DNA at a specific nucleotide sequence. The cutting or cleavage site may be the same as or different from the recognition sequence. A “restriction enzyme site” is a nucleotide sequence that targets cleavage of the DNA by a restriction enzyme. It is recognized that a variety of restriction enzymes will be useful in the invention, the choice of which will depend in part on the RNA source organism and the frequency of the restriction enzyme site's occurrence. Restriction enzyme sites occur with varying frequency in natural nucleotide sequences. Typically as the complexity of the restriction enzyme site nucleotide sequence increases, the frequency with which the restriction enzyme site occurs decreases. In an embodiment, splint oligonucleotides comprise a restriction enzyme site. The restriction enzyme site utilized in the splint oligonucleotide may occur in the source genome with high, medium, or low frequency. By low frequency is intended at levels of about 1/1000 transcripts to about 1/100,000 transcripts to about 1/500,000 transcripts.

The amplified cDNA is transcribed into amplified RNA. In an embodiment, products of the rolling circle amplification are labeled using a variety methods including, but not limited to, in vitro transcription. Methods of performing in vitro transcription are known in the art. Components for optimizing in vitro transcription are known in the art. See, for example, Ausubel et al., eds. (2003) Current Protocols in Molecular Biology, (John Wiley & Sons, New York) and Sambrook et al. (2001) Molecular Cloning: A Laboratory Manual (3d ed., Cold Spring Harbor Laboratory Press, Plainview, N.Y., herein incorporated by reference in their entirety.

Transcripts within the amplified RNA samples which represent RNA produced by differentially expressed genes may be identified by utilizing a variety of methods which are well known to those of skill in the art (Bowtell & Sambrook Eds. (2003) DNA Microarrays (Cold Spring Harbor Laboratory Press, Cold Spring Harbor, N.Y., herein incorporated by reference in its entirety). For example, differential screening (Tedder, T. F. et al., 1988, Proc. Natl. Acad. Sci. USA 85:208-212), subtractive hybridization (Hedrick, S. M. et al., 1984, Nature 308:149-153; Lee, S. W. et al., 1984, Proc. Natl. Acad. Sci. USA 88:2825), and, differential display (Liang, P., and Pardee, A. B., 1993, U.S. Pat. No. 5,262,311, incorporated herein by reference in their entirety), may be utilized to identify nucleic acid sequences derived from genes that are differentially expressed.

“Differential expression” as used herein refers to both quantitative as well as qualitative differences in the genes' temporal and/or tissue expression patterns. Thus, a differentially expressed gene may have its expression activated or completely inactivated in normal versus disease conditions (e.g., treated versus untreated), or under control versus experimental conditions. Such a qualitatively regulated gene will exhibit an expression pattern within a given tissue or cell type which is detectable in either control or disease subjects, but is not detectable in both. Alternatively, such a qualitatively regulated gene will exhibit an expression pattern within a given tissue or cell type which is detectable in either control or experimental subjects, but is not detectable in both. “Detectable”, as used herein, refers to an RNA expression pattern which is detectable via the standard techniques of differential display, hybridization, reverse transcriptase- (RT-) PCR and/or Northern analyses, which are well known to those of skill in the art or by the methods of the invention.

Alternatively, a differentially expressed gene may have its expression modulated, i.e., quantitatively increased or decreased, in normal versus disease states, or under control versus experimental conditions. Transcript levels of differentially expressed genes may vary by 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100% or more.

In an embodiment the amplified RNA is contacted with an ordered matrix of hybridization probes, under hybridizing conditions. The probes are generally immobilized and arrayed onto a solid substrate. The amplified RNA can be hybridized to high density arrays containing hundreds or thousands of oligonucleotide probes (Cronin, M. T. et al. (1996) Human Mutation 7: 244-255; Kozal, M. J. et al. (1996) Nature Medicine 2: 753-759, herein incorporated by reference in their entirety. A hybridization signal is then detected at each hybridization pair to obtain a transcription signal profile. A wide variety of hybridization signals may be used. In one embodiment, the amplified RNA is labeled with radionucleotides such that the amplified RNA provides a radioactive signal that can be detected in the hybridization pairs. In an embodiment, the RNA is amplified with a fluorescently tagged nucleotide. In an embodiment, the RNA is amplified with a biotin labeled nucleotide which can then specifically bind fluorescently labeled streptavidin. The transcription signal profile for each sample is compared.

By “probe” is intended an isolated nucleic acid molecule corresponding to a nucleotide sequence of interest. A probe may be any one of a collection of many isolated nucleic acid molecules of differing nucleotide sequences. The collection of nucleic acid molecules may be in solution, adhered to a membrane, embedded on a microchip, embedded on an array, or adhered to a support structure.

In an embodiment of the invention, synthesizing cDNA comprises a first strand synthesis step and a second strand synthesis step. First strand synthesis is performed as described elsewhere herein. Reverse transcriptase extends the primer to generate an RNA-cDNA mixture. The cDNA has known, predetermined sequence at the 5′ ends introduced by the primer. In an embodiment, the RNA and cDNA mixture is incubated with a ribonuclease H. In an embodiment the RNA and cDNA mixture is incubated with a 3′ to 5′ exonuclease. In an embodiment, the RNA and cDNA mixture is incubated with a ribonuclease H and a 3′ to 5′ exonuclease. Second strand synthesis reaction components are incubated with the first strand of cDNA. Second strand synthesis yields double-stranded cDNA.

By “second strand synthesis” is intended the polymerization of a DNA strand complementary to the first cDNA strand. Methods of priming second strand synthesis are known in the art. Second strand synthesis is primed with a variety of primer types including, but not limited to, degraded remnants of the RNA strand, random oligonucleotide primers, such as random hexamers, oligonucleotide primers of known nucleotide sequence, and hairpin structures. A DNA-dependent DNA polymerase extends the primer or primers on the first strand to generate the second strand. DNA polymerases are known in the art and include, but are not limited to, E. coli DNA polymerase I, bacteriophage T4 DNA polymerase, and the Klenow fragment. The product of second strand synthesis is a double-stranded cDNA molecule.

Typical second strand synthesis reaction components include, but are not limited to, Tris-HCl, KCl, MgCl₂, β-NAD, DTT, (NH₄)₂SO₄, dATP, dTTP, dCTP, dGTP, ligase, BSA, ribonuclease H, T4 DNA polymerase, and Klenow. The Tris-HCl concentration in a second-strand synthesis reaction may range from 0 to 400 mM, preferably from 0.4 to 100 mM, more preferably from 1 to 40 mM. Such concentrations include, but are not limited to, 0, 0.1, 0.2, 0.4, 0.6, 0.8, 1, 2, 4, 6, 8, 10, 15, 20, 25, 30, 35, 40, 45, 50, 55, 60, 65, 70, 75, 80, 85, 90, 95, 100, 150, 200, 250, 300, 350, and 400 mM Tris-HCl. The Tris-HCl pH in a second strand synthesis reaction may range from pH 6.5 to 8.8, preferably from 6.9 to 8.0, more preferably from 7.2 to 8.0. Such pHs include, but are not limited to, 6.5, 6.7, 6.8 6.9, 7.2, 7.5, 7.8, 8.0, 8.3, 8.5, and 8.8. The KCl concentration in a second strand synthesis reaction may range from 0 to 450 mM, preferably from 0.1 to 220 mM, more preferably from 1 to 100 mM. Such concentrations include, but are not limited to, 0, 0.1, 0.9, 1, 2, 3, 4, 5, 6, 7, 8, 9, 10, 20, 30, 40, 50, 60, 70, 75, 80, 85, 90, 95, 100, 120, 140, 160, 180, 200, 220, 250, 300, 350, 400, and 450 mM KCl. The MgCl₂ concentration in a second strand synthesis reaction may range from 0 to 100 mM, preferably from 0.01 to 50 mM, more preferably from 0.1 to 10 mM. Such concentrations include, but are not limited to, 0, 0.01, 0.1, 0.2, 0.3, 0.4, 0.5, 0.6, 0.7, 0.8, 0.9, 1, 2, 3, 4, 4.06, 5, 6, 7, 8, 9, 10, 11, 12, 13, 14, 15, 16, 17, 18, 19, 20, 25, 30, 40, 50, 60, 70, 80, 90, and 100 mM MgCl₂. The β-NAD concentration in a second strand synthesis reaction may range from 0 to 300 μM, preferably from 10 to 200 μM, more preferably from 30 to 150 μM. Such concentrations include, but are not limited to, 0, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, and 300 μM β-NAD. The dithiothreitol concentration in a second strand synthesis reaction may range from 0 to 75 mM, preferably 0.1 to 20 mM, more preferably from 1 to 10 mM DTT. Such concentrations include, but are not limited to, 0, 0.1, 1, 2, 3, 4, 5, 6, 7, 8, 9, 10, 11, 12, 13, 14, 15, 16, 17, 18, 19, 20, 25, 30, 40, 50, 60, 70, and 75 mM dithiothreitol. The (NH₄)₂SO₄ concentration in a second strand synthesis reaction may range from 0 to 100 mM, preferably 0.1 to 50 mM, more preferably 1 to 25 mM. Such concentrations include, but are not limited to, 0, 0.1, 1, 2, 3, 4, 5, 6, 7, 8, 9, 10, 11, 12, 13, 14, 15, 16, 17, 18, 19, 20, 25, 30, 40, 50, 60, 70, 80, 90, to 100 mM (NH₄)₂SO₄. The dATP concentration in a second strand synthesis reaction may range from 0.01 to 330 μM, preferably from 0.1 to 100 μM, more preferably from 1 to 33 μM. Such concentrations include, but are not limited to, 0.01, 0.05, 0.1, 0.5, 1, 2, 3, 4, 5, 6, 7, 8, 9, 10, 15, 20, 25, 30, 33, 35, 40, 45, 50, 55, 60, 65, 70, 75, 80, 85, 90, 100, 110, 120, 130, 140, 150, 160, 170, 180, 190, 200, 210, 220, 230, 240, 250, 260, 270, 280, 290, 300, 310, 320, and 330 μM dATP. The dGTP concentration in a second strand synthesis reaction may range from 0.01 to 330 μM, preferably from 0.1 to 100 μM, more preferably from 1 to 33 μM. Such concentrations include, but are not limited to, 0.01, 0.05, 0.1, 0.5, 1, 2, 3, 4, 5, 6, 7, 8, 9, 10, 15, 20, 25, 30, 33, 35, 40, 45, 50, 55, 60, 65, 70, 75, 80, 85, 90, 100, 110, 120, 130, 140, 150, 160, 170, 180, 190, 200, 210, 220, 230, 240, 250, 260, 270, 280, 290, 300, 310, 320, and 330 μM dGTP. The dCTP concentration in a second strand synthesis reaction may range from 0.01 to 330 μM, preferably from 0.1 to 100 μM, more preferably from 1 to 33 μM. Such concentrations include, but are not limited to, 0.01, 0.05, 0.1, 0.5, 1, 2, 3, 4, 5, 6, 7, 8, 9, 10, 15, 20, 25, 30, 33, 35, 40, 45, 50, 55, 60, 65, 70, 75, 80, 85, 90, 100, 110, 120, 130, 140, 150, 160, 170, 180, 190, 200, 210, 220, 230, 240, 250, 260, 270, 280, 290, 300, 310, 320, and 330 μm dCTP. The dTTP concentration in a second strand synthesis reaction may range from 0.01 to 330 μM, preferably from 0.1 to 100 μM, more preferably from 1 to 33 μM. Such concentrations include, but are not limited to, 0.01, 0.05, 0.1, 0.5, 1, 2, 3, 4, 5, 6, 7, 8, 9, 10, 15, 20, 25, 30, 33, 35, 40, 45, 50, 55, 60, 65, 70, 75, 80, 85, 90, 100, 110, 120, 130, 140, 150, 160, 170, 180, 190, 200, 210, 220, 230, 240, 250, 260, 270, 280, 290, 300, 310, 320, and 330 μM dTTP. The ligase concentration in a second strand synthesis reaction may range from 0 to 10 U/15 μl, preferably 0.01 to 10 U/15 μl, more preferably from 0.1 to 10 U/15 μl. Such concentrations include, but are not limited to, 0, 0.01, 0.1, 1, and 10 U/15 μl. The acetylated bovine serum albumin (BSA) concentration in a second strand synthesis reaction may range from 0 to 100 μg/μl, preferably from 0.01 to 10 μg/μl, more preferably from 0.1 to 10 μg/μl. Such concentrations include, but are not limited to, 0, 0.01, 0.1, 1, 10, and 100 μg/μl. The ribonuclease H concentration in a second synthesis reaction may range from 0 to 10 U/100 μl, preferably from 0.01 to 10 U/100 μl, more preferably from 0.1 to 20 U/100 μl. Such concentrations include, but are not limited to 0, 0.01, 0.1, 0.2, 0.3, 0.4, 0.5, 0.6, 0.7, 0.8, 0.9, 1, 2, 3, 4, 5, 6, 7, 8, 9, and 10 U/100λ. The T4DNA polymerase concentration in a second strand synthesis reaction may range from 0 to 100 U/100 μl, preferably from 0.1 to 50 U/100 μl, more preferably from 1 to 10 U/100 μl. Such concentrations include, but are not limited to 0, 0.1, 1, 2, 3, 4, 5, 6, 7, 8, 9, 10, 15, 20, 25, 30, 35, 40, 45, 50, 55, 60, 65, 70, 75, 80, 90, to 100 U/100 μl. The Klenow fragment concentration in a second strand synthesis reaction may range from 0 to 10 U/1 μg DNA, preferably from 0.01 to 10 U/1 μg DNA, more preferably from 0.1 to 10 U/μg DNA. Such concentrations include, but are not limited to 0, 0.01, 0.1, 1, to 10 U Klenow/1 μg DNA.

Reaction conditions for second strand synthesis are known in the art. Second strand reaction incubations include, but are not limited to, incubations at temperatures ranging from 4° C., 6° C., 8° C., 10° C., 12° C., 14° C., 16° C., 18° C., 20° C., 22° C., 24° C., 25° C., 26° C., 28° C., 30° C., 35° C., 37° C., 40° C., 45° C., 50° C., 55° C., 60° C., 65° C., 70° C., 75° C., 80° C., 85° C., to 90° C. Durations of incubation periods for second strand synthesis reactions range from 0, 1, 5, 10, 20, 30, 40, 50, 60, 70, 80, 90, 100, 110, 120, 130, 140, 150, 160, 170, 180, 190, 200, 210, 220, 230, to 240 minutes or more. Second strand synthesis reaction may undergo multiple incubation periods or multiple cycles of incubation periods. See, for example, Ausubel et al., eds. (2003) Current Protocols in Molecular Biology, (John Wiley & Sons, New York) and Sambrook et al. (2001) Molecular Cloning: A Laboratory Manual (3d ed., Cold Spring Harbor Laboratory Press, Plainview, N.Y., herein incorporated by reference.

In the embodiment, a terminal transferase is incubated with linear double-stranded cDNA. Terminal transferase modifies the 3′ termini of cDNA by adding a first homopolymer region, as described elsewhere herein. After the terminal transferase reaction, linear double-stranded cDNA have a first homopolymer region at the 3′ termini of the first and second strands. After the terminal transferase incubation both ends of the double-stranded cDNA molecules have predetermined nucleotide sequence although it is recognized that the nucleotide sequence at the 5′ end of the second strand may be unknown.

The double-stranded cDNA is incubated with a splint oligonucleotide phosphorylated at the 5′ end. Splint oligonucleotides are described elsewhere herein. In an embodiment, the nucleotide sequence of the splint oligonucleotide is set forth in SEQ ID NO:2. In an embodiment the nucleotide sequence of the splint oligonucleotide is set forth in SEQ ID NO:8. In an embodiment splint oligonucleotides anneal to the single stranded 3′ ends of double-stranded cDNA, and the splint oligonucleotides anneal to each other (FIG. 2). In an embodiment, splint oligonucleotides anneal to each other forming a double-stranded molecule with 3′ single-stranded regions. The 3′ single-stranded regions anneal to the 3′ ends of double-stranded cDNA (FIG. 3). In an embodiment, splint oligonucleotides link the 5′ and 3′ ends of multiple cDNA molecules. The second homopolymer region of the splint oligonucleotides anneals to the first homopolymer region on the cDNA ends. The variable regions of the first and second splint oligonucleotides anneal to each other, thus circularizing the cDNA. Alternatively, the variable regions of the first and second splint oligonucleotides anneal to each other, then the second homopolymer regions anneal to the first homopolymer regions on the cDNA.

As described elsewhere herein, the splint oligonucleotides anneal to the cDNA ends, bringing the ends in close proximity for ligation. A gap-filling polymerase incorporates bases to fill-in gaps in the strands. Circular cDNA molecules suitable for rolling circle amplification are formed upon ligation. The circularized cDNA is incubated with a rolling circle DNA polymerase and rolling circle replication reaction components. In an embodiment, the amplified cDNA is incubated with a restriction enzyme. The amplified cDNA is transcribed into amplified RNA.

Compositions of the invention include a kit providing reagents for RNA amplification through a method of the invention. A kit of the invention includes, but is not limited to, primer, reverse transcriptase, terminal transferase, a deoxynucleotide triphosphate solution, splint oligonucleotide, ligase, and rolling circle amplification components. These reagents are described elsewhere herein. A kit of the invention may also include RNA isolation components, cDNA synthesis reaction components, ribonuclease H reaction components, 3′ to 5′ exonuclease reaction components, terminal transferase reaction components, cDNA purification components, annealing components, ligase reaction components, a restriction enzyme and restriction enzyme reaction components, or in vitro transcription components. Typical RNA amplification reagents are described elsewhere herein

By “deoxynucleoside triphosphate solution” is intended a solution comprising either deoxyadenosine triphosphate, deoxyguanosine triphosphate, deoxycytidine triphosphate, or thymidine triphosphate. The deoxynucleoside triphosphate concentration of a deoxynucleoside triphosphate solution provided in a kit of the invention may be at or above the concentration normally used in a particular reaction. The concentration of the deoxynucleoside triphosphate may range from 0.01 to 400 mM, preferably from 0.1 to 200 mM, more preferably from 1 to 100 mM. Such concentrations include, but are not limited to, 0.01, 0.05, 0.1, 0.5, 1, 2, 3, 4, 5, 6, 7, 8, 9, 10, 15, 20, 25, 30, 33, 35, 40, 45, 50, 55, 60, 65, 70, 75, 80, 85, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, and 400 mM.

It is in the scope of the present invention that the rolling circle amplification products may be used for diagnostic and monitoring purposes. Relative abundance of different transcripts associated with disease progression may be analyzed using the rolling circle amplification products.

Diseases of interest include, but are not limited to, cancers, carcinomas, sarcomas, congenital disorders, bacterial, viral, and fungal diseases. Of particular interest are those diseases for which only small samples of tissue are available or for which it is undesirable to obtain large tissue samples. Such diseases include, but are not limited to, kidney disorders, such as focal segmental glomeuorosclerosis; brain disorders; nervous system disorders; reproductive disorders; fetal disorders; cardiovascular disorders, lymphatic disorders, gastrointestinal disorders, respiratory system disorders, skin disorders, and glandular disorders.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

It is envisioned that the rolling circle amplification products may be used to generate representational libraries. Such representational libraries would reflect the relative abundance of different transcripts in the original source.

The following examples are offered by way of illustration and not limitation.

### EXPERIMENTAL

### Example 1

### Synthesis of Double-Strand cDNA

Total RNA was isolated using the Nanoprep™ kit (Stratagene). The (dT)-T7 primer (SEQ ID NO:1) was HPLC purified then treated with kinase. The (dT)-T7 primer was incubated with 100 pg total RNA. A reverse transcription reaction was performed by the addition of 5× First Strand Buffer, 100 mM DTT, 10 mM dNTP, T4gp32, RNase Inhibitor, and Superscript II reverse transcriptase. The dNTP solution contained equimolar amounts of the four nucleotides. The reaction mixture was incubated. Second strand synthesis was performed by the addition of 5× Second Strand Buffer, 10 mM dNTP, DNA Polymerase I, 1 U E. coli RNAse H, E. coli DNA ligase, and T4 DNA polymerase. The reaction mixture was incubated. The reaction mixture was treated with Exonuclease I. The double-stranded cDNA was purified using the Microcon YM-100 system from Millipore.

### Example 2

### Addition of polyA Tails by Terminal Transferase

10× buffer, 25 mM CoCl₂, 10 mM dATP, and terminal transferase were added to the cDNA such that the concentration of these components in the terminal transferase reaction was 1× buffer, 2.5 mM CoCl₂, and 1.0 mM dATP. The reaction mixtures were incubated. The cDNA was purified using the Microcon YM-100 system from Millipore.

### Example 3

### Circularization of the cDNA by a Splint Oligonucleotide

T tail primer (SEQ ID NO:2), a splint oligonucleotide, was incubated with kinase. The T tail primer annealed to the polyA tailed, double-stranded cDNA. 10× buffer, 1.0 μl 1 mM dTTP, Klenow fragment, and T4 ligase were added to the reaction components. The reactions were incubated. The cDNA was purified using the Microcon YM-100 system from Millipore.

### Example 4

### Rolling Circle Amplification of the Circularized cDNA

The circularized cDNA was incubated with Φ29 DNA polymerase and rolling circle amplification reaction components as recommended by the Φ29 DNA polymerase supplier (Amersham Bioscience, Inc.) The products of the reaction were incubated with AscI in 10× buffer diluted to a final 1× concentration. The cDNA was purified using the DNA Clean Kit system (Zymo Research).

### Example 5

### In Vitro Transcription of the Amplified cDNA

In vitro transcription was performed using the ENZO™ kit. Five microliters of amplified cDNA was placed in a 1.5 ml microfuge tube. The volume was brought to 40 microliters by the addition of nuclease free water. Concentrated reaction buffer, biotin nucleotides, DTT, RNase inhibitor, and T7 RNA polymerase were added to the cDNA according to standard protocols. The reaction components were mixed and incubated at 37° C. for 4-5 hours. The amplified RNA was purified using Qiagen™ columns.

### Example 6

### Preparation of Circularized cDNA with Hairpin Primer

Total RNA was isolated using the Nanoprep™ kit (Stratagene). The hairpin primer (SEQ ID NO:6) was HPLC purified then treated with kinase. The hairpin primer was incubated with 10 ng total RNA. A reverse transcription reaction was performed by the addition of 5× First Strand Buffer, 100 mM DTT, 10 mM dNTP, T4gp32, RNase Inhibitor, and Superscript II reverse transcriptase. The dNTP solution contained equimolar amounts of the four nucleotides. The reaction mixture was incubated. The reaction mixture was incubated with Exonuclease I. The reaction mixture was incubated with RNAase H. Terminal transferase was used to add poly(dA) tails (described above). An annealing incubation was performed wherein the cDNA was incubated under conditions suitable for intramolecular annealing to allow the poly(A) to anneal to the poly(dT) region. The looped cDNA was incubated with DNA polymerase to fill the gaps and ligase to ligate the 5′ and 3′ ends. The circularized cDNA was then amplified by rolling circle amplification.

All publications, patents, and patent applications mentioned in the specification are indicative of the level of those skilled in the art to which this invention pertains. All publications, patents, and patent applications are herein incorporated by reference to the same extent as if each individual publication or patent application was specifically and individually incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method of amplifying RNA, said method comprising:
(a) providing RNA;
(b) synthesizing cDNA with predetermined nucleotide sequences at the 3′ end of the cDNA from said RNA;
(c) incubating said cDNA with a splint oligonucleotide;
(d) circularizing said cDNA; and
(e) preparing amplified cDNA from the circularized cDNA using rolling circle replication.

2. The method of claim 1, wherein said RNA is incubated with a primer.

3. The method of claim 2, wherein said primer provides predetermined nucleotide sequence at the 5′ end of the cDNA.

4. The method of claim 2, wherein said primer is an isolated nucleic acid molecule comprising a poly(dT) region.

5. The method of claim 4, wherein said poly(dT) region comprises at least 5 thymidine residues.

6. The method of claim 2, wherein said primer is an isolated nucleic acid molecule comprising a promoter region.

7. The method of claim 6, wherein said promoter region comprises a nucleotide sequence selected from the group consisting of:
(a) the nucleotide sequence set forth in SEQ ID NO:3;
(b) the nucleotide sequence set forth in SEQ ID NO:4; and
(c) a nucleotide sequence having at least about 90% sequence identity to a nucleotide sequence set forth in SEQ ID NO:3 or SEQ ID NO:4, wherein said nucleotide sequence is capable of initiating transcription.

8. The method of claim 1, wherein said primer is an isolated nucleic acid molecule comprising a nucleotide sequence selected from the group consisting of:
(a) a nucleotide sequence having the nucleotide sequence set forth in SEQ ID NO:1; and
(b) a nucleotide sequence having the nucleotide sequence set forth in SEQ ID NO:7.

9. The method of claim 1, wherein synthesizing cDNA comprises the step of first strand synthesis.

10. The method of claim 1, wherein synthesizing cDNA comprises the steps of first strand synthesis and second strand synthesis.

11. The method of claim 1, wherein synthesizing cDNA comprises incubating the RNA with a reverse transcriptase.

12. The method of claim 1, wherein synthesizing cDNA comprises incubating a ribonuclease H with the RNA and cDNA.

13. The method of claim 1, wherein incubating a terminal transferase with said cDNA provides said predetermined nucleotide sequence at a 3′ end of the cDNA.

14. The method of claim 13, wherein said terminal transferase modifies a 3′ terminus of said cDNA by adding a first homopolymer region.

15. The method of claim 14, wherein said first homopolymer region comprises at least 5 homogenous nucleosides.

16. The method of claim 15, wherein the nucleoside of said first homopolymer region is selected from the group consisting of: deoxyadenosine, deoxyguanosine, deoxycytidine, and thymidine.

17. The method of claim 16, wherein the nucleoside of said first homopolymer region is deoxyadenosine.

18. The method of claim 16, wherein the nucleoside of said first homopolymer region is deoxycytidine.

19. The method of claim 1, wherein said splint oligonucleotide is an isolated nucleic acid molecule comprising a second homopolymer region and a variable region.

20. The method of claim 19, wherein said second homopolymer region comprises the nucleoside complementary to the nucleoside of the first homopolymer region.

21. The method of claim 19, wherein the nucleoside of said second homopolymer region is thymidine.

22. The method of claim 19, wherein the nucleoside of said second homopolymer region is deoxyguanosine.

23. The method of claim 19, wherein said second homopolymer region is at least 5 nucleotides.

24. The method of claim 19, wherein the nucleotide sequence of the variable region comprises a palindrome.

25. The method of claim 19, wherein the nucleotide sequence of the variable region comprises a restriction enzyme site.

26. The method of claim 19, wherein said variable region comprises a primer complementary region and a central region.

27. The method of claim 26, wherein said central region comprises a promoter region.

28. The method of claim 1, wherein said splint oligonucleotide is an isolated nucleic acid molecule comprising a nucleotide sequence selected from the group consisting of:
(a) a nucleotide sequence having the sequence set forth in SEQ ID NO:2; and
(b) a nucleotide sequence having the sequence set forth in SEQ ID NO:8.

29. The method of claim 1, wherein the splint oligonucleotide anneals to the 5′ and 3′ ends of linear cDNA.

30. The method of claim 29, wherein annealing said splint oligonucleotide circularizes said cDNA.

31. The method of claim 1, wherein a splint oligonucleotide anneals to the 3′ end of linear cDNA.

32. The method of claim 31, wherein annealing said splint oligonucleotide circularizes said cDNA.

33. The method of claim 31, wherein said cDNA is single-stranded.

34. The method of claim 31, wherein said cDNA is double-stranded.

35. The method of claim 34, wherein a first and a second splint oligonucleotide anneal to the first and second 3′ ends of said double-stranded cDNA.

36. The method of claim 35, wherein the variable regions of said first and second splint oligonucleotides anneal.

37. The method of claim 36, wherein annealing said variable regions circularizes said cDNA.

38. The method of claim 1, comprising incubating said cDNA with a 3′ to 5′ exonuclease.

39. The method of claim 38, wherein said 3′ to 5′ exonuclease is Exonuclease I.

40. The method of claim 1, comprising incubating a DNA ligase with the circularized cDNA prior to amplifying the circularized cDNA.

41. The method of claim 1, comprising transcribing the amplified cDNA into amplified RNA.

42. The method of claim 41, comprising incubating the cDNA with a restriction enzyme prior to transcribing the amplified cDNA.

43. A kit for amplifying RNA comprising a primer, a reverse transcriptase, a terminal transferase, a deoxynucleoside triphosphate solution, a splint oligonucleotide, a gap-filling polymerase, a ligase, and rolling circle amplification reaction components, wherein said splint oligonucleotide is an isolated nucleic acid molecule having a nucleotide sequence comprising a sequence selected from the group consisting of:
(a) a nucleotide sequence having the nucleotide sequence set forth in SEQ ID NO:2; and
(b) a nucleotide sequence having the nucleotide sequence set forth in SEQ ID NO:8.

44. A method for comparing RNA expression levels in multiple samples, said method comprising:
(a) providing RNA from multiple samples;
(b) synthesizing cDNA with predetermined nucleotide sequences at a 3′ end of the cDNA from said RNA;
(c) incubating said cDNA with a splint oligonucleotide;
(d) circularizing said cDNA;
(e) preparing amplified cDNA from the circularized cDNA using rolling circle replication; and
(f) evaluating RNA expression levels.

45. The method of claim 44 comprising the steps of:
(a) transcribing the amplified cDNA from each sample;
(b) incubating the amplified RNA from each sample with a probe; and
(c) analyzing the results.

46. The method of claim 45, wherein said probe is located on a microarray.

47. The method of claim 45, wherein said probe is in solution.

48. A method for amplifying RNA obtained from a subject, comprising the steps of:
(a) converting the RNA into circularized cDNA suitable for amplification by rolling circle replication;
(b) amplifying the cDNA using rolling circle replication; and
(c) transcribing the amplified cDNA into RNA, resulting in amplified RNA.

49. A method of amplifying RNA, said method comprising:
(a) providing RNA;
(b) synthesizing cDNA with predetermined nucleotide sequences at the 5′ and 3′ ends of the cDNA from said RNA;
(c) circularizing said cDNA; and
(d) preparing amplified cDNA from the circularized cDNA using rolling circle replication.

50. The method of claim 49, wherein said RNA is incubated with a primer.

51. The method of claim 50, wherein said primer is an isolated nucleic acid molecule comprising a hairpin region, a promoter region, and a poly(dT) region.

52. The method of claim 50, wherein said primer is an isolated nucleic acid molecule having a nucleotide sequence comprising the nucleotide sequence set forth in SEQ ID NO:6.

53. The method of claim 49, wherein synthesizing cDNA comprises the step of first strand synthesis.

54. The method of claim 49, wherein synthesizing cDNA comprises incubating the RNA with a reverse transcriptase.

55. The method of claim 49, comprising incubating said cDNA with a 3′ to 5′ exonuclease.

56. The method of claim 55, wherein said 3′ to 5′ exonuclease is Exonuclease I.

57. The method of claim 49, wherein synthesizing cDNA comprises incubating a ribonuclease H with the RNA and cDNA.

58. The method of claim 49, comprising the step of isolating said cDNA.

59. The method of claim 49, wherein incubating a terminal transferase with said cDNA provides said predetermined nucleotide sequence at the 3′ end of the cDNA.

60. The method of claim 59, wherein said terminal transferase modifies a 3′ terminus of said cDNA by adding a first homopolymer region.

61. The method of claim 60, wherein said first homopolymer region comprises at least 5 homogenous nucleotides.

62. The method of claim 60, wherein the nucleoside of said first homopolymer region is deoxyadenosine.

63. The method of claim 49, comprising an annealing incubation step.

64. The method of claim 62, wherein said first homopolymer region anneals with said poly(dT) region.

65. The method of claim 64, wherein said annealing circularizes said cDNA.

66. The method of claim 49, comprising incubating a DNA ligase with the circularized cDNA prior to amplifying the circularized cDNA.

67. The method of claim 49, wherein amplifying the circularized cDNA using rolling circle replication comprises the step of incubating a rolling circle DNA polymerase with said circularized cDNA.

68. The method of claim 49, comprising incubating amplified cDNA with a restriction enzyme.

69. The method of claim 49, comprising transcribing the amplified cDNA into amplified RNA.

70. A kit for amplifying RNA comprising a primer, a reverse transcriptase, a terminal transferase, a deoxynucleoside triphosphate solution, a ligase, and rolling circle amplification reaction components, wherein said primer is an isolated nucleic acid molecule comprising a hairpin region, a promoter region, and a poly(dT) region.

71. The kit of claim 70, wherein said primer is an isolated nucleic acid molecule having a nucleotide sequence comprising the nucleotide sequence set forth in SEQ ID NO:6.

72. A method for comparing RNA expression levels in multiple samples, said method comprising:
(a) providing RNA from multiple samples;
(b) synthesizing cDNA with predetermined nucleotide sequences at the 5′ and 3′ ends of the cDNA from said RNA;
(c) circularizing said cDNA;
(d) preparing amplified cDNA from the circularized cDNA using rolling circle replication; and
(e) evaluating RNA expression levels.

73. The method of claim 72, wherein said RNA is incubated with a primer.

74. The method of claim 73, wherein said primer comprises a hairpin region.

75. The method of claim 72, comprising incubating said cDNA with a 3′ to 5′ exonuclease.

76. The method of claim 72 comprising the steps of:
(a) transcribing the amplified cDNA from each sample into amplified RNA;
(b) incubating said amplified RNA from each sample with a probe; and
(c) analyzing the results.
